(19) United States
(12) Patent Application Publication
(10) Pub. No.: US 2024/0277969 A1
(43) Pub. Date: Aug. 22, 2024

(54) Locomotion-Assisting Method and Apparatus

(71) Applicant: GAITAR LIMITED, Lossiemouth, Scotland (GB)

(72) Inventor: William Scott Christie McLeod, Scotland (GB)

(21) Appl. No.: 18/681,144

(22) PCT Filed: Aug. 2, 2022
(86) PCT No.: PCT/GB2022/052035

§ 371 Date: Feb. 5, 2024

Related U.S. Application Data
(60) Provisional application No. 63/230,710, filed on Aug. 7, 2021.

(30) Foreign Application Priority Data
Mar. 3, 2022 (GB) 2202944.1

(51) Int. Cl. A61M 21/00

(52) U.S. Cl. CPC A61M 21/00; A61M 2021/0022; A61M 2021/005

(57) ABSTRACT

Locomotion-assisting apparatus and method of assisting an individual with impaired locomotion from a neurodegenerative disease such as Parkinson's Disease, so as to improve the individual's locomotion, comprising mounting to the individual apparatus comprising a projection device and a display means and projecting an image from the projecting device on to the display means, the image being perceived by the individual to be in an upper field of view and perceived to be a distance in front of the individual.

The present invention relates to improvements in locomotion patterns and/or gait of individuals. In particular, the present invention seeks to improve the walking pattern and/or gait of patients with neurodegenerative diseases such as Parkinson's Disease. Parkinson's is the fastest-growing neurodegenerative condition, with its prevalence set to rise by fifty percent by the year 2030, yet a cure remains elusive. People with Parkinson's Disease develop changes in their walking from diagnosis, which results in the adoption of a shuffling walking pattern which predisposes them to increased risk of falls, physical deconditioning, and reduced quality of life.

It is believed there are around 12 million people globally who are presently living with Parkinson's Disease with that number set to grow at an alarming rate. 1 in 37 people will get Parkinson's in their lifetime, and 1 in 20 people will be under the age of 40. Medication has been used to treat the symptoms of Parkinson's Disease, but at this time there remains no cure for this debilitating disease.

The main symptoms of Parkinson's Disease are:
Involuntary shaking
Muscles stiffness
Shuffling walk
Slowness of movement
Freezing of gait (FOG)—this is when the person can't move and feels like they are stuck to the floor

Treatments for Parkinson's Disease include therapies to help with movement problems, physical activity, medicines, and sometimes brain surgery.

A person with Parkinson's Disease can also experience a wide range of other physical and psychological symptoms, and these include depression and anxiety, balance problems, loss of sense of smell, memory problems, insomnia, and constipation. An effective treatment to effectively manage changes in walking and/or gait is required.

Visual cueing is a method that has been used in physiotherapy clinics all over the World and involves marking out a pathway on the floor in front of the patient using markers such as lines of tape or pieces of paper, and when the patient walks over these markers, they display an instant improvement in stride length and speed. It is believed that flexible motor control is enabled by signals sent from the basal ganglia which is triggered by dopamine. People with Parkinson's Disease lose the ability to create dopamine which is the cause of their mobility symptoms. Visual cueing works by by-passing the “basal ganglia” and sends a signal directly to the flexible motor control part of the brain instantly improving mobility.

However, despite being helpful, these known visual cues still cause a stooped-over stance and stumbling so that sufferers of Parkinson's Disease are still self-conscious of their condition and how they appear different from other people.

According to one aspect of the present invention, there is provided locomotion-assisting apparatus comprising display means, mounting means for mounting said display means on a user and a projection device serving to project an image onto the display means in an upper region of the display means, the image perceived by the user to appear in an upper field of view of the user and to be a distance in front of the user.

According to a second aspect of the present invention, there is a method of assisting an individual with impaired locomotion, so as to improve the individual's locomotion, comprising mounting to the individual apparatus comprising a projection device and a display means and projecting an image from the projecting device on to the display means, the image being perceived by the individual to be in an upper field of view and perceived to be a distance in front of the individual.

According to a third aspect of the present invention, there is provided a method of assisting people with neurodegenerative disease by utilizing a heads-up display module on a user and projecting a hologram-type image to appear in an upper field of view of the user and to be a distance in front of the user.

Owing to these aspects, the projected image, as perceived by the individual user suffering from a neurodegenerative disease, can dramatically change, for the better, their locomotion.

The display means is preferably a transparent display means. This allows the user to see what is shown on the display means while still being able to see through the display means.

The projecting device and display means advantageously provide an optical display system.

Head-mounted optical display systems are a preferred embodiment in the form of an eyeglass or pair of eyeglasses that form a heads-up-display (HUD) module to be worn by the user.

The projected image is preferably a very simple image which is advantageously static, such as a horizontal line that appears in the upper field of view of the user and focused to infinity.

In order that the present invention can be clearly and completely disclosed, reference will now be made, by way of example only, to the accompanying drawings, in which:—

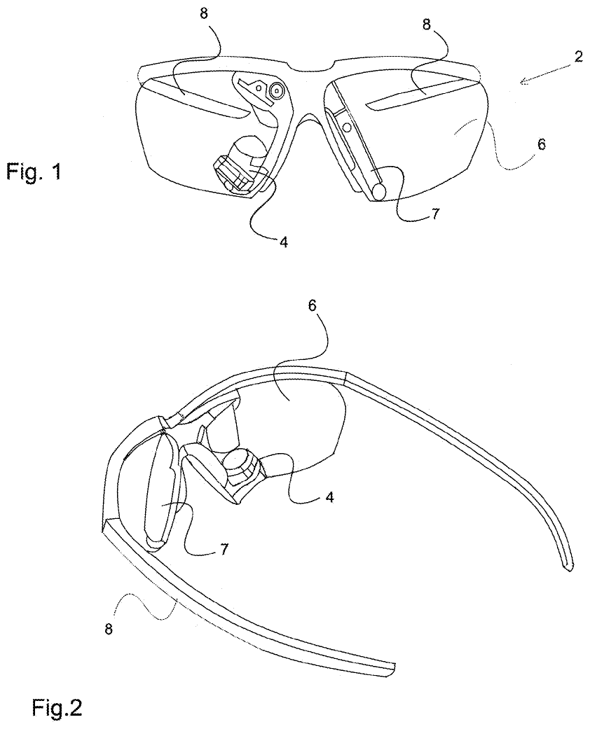

FIG. 1 shows a front view of a heads-up-display module for improving the locomotion of an individual who suffers locomotive problems caused by Parkinson's disease,

FIG. 2 shows a rear perspective view of the module of FIG. 1,

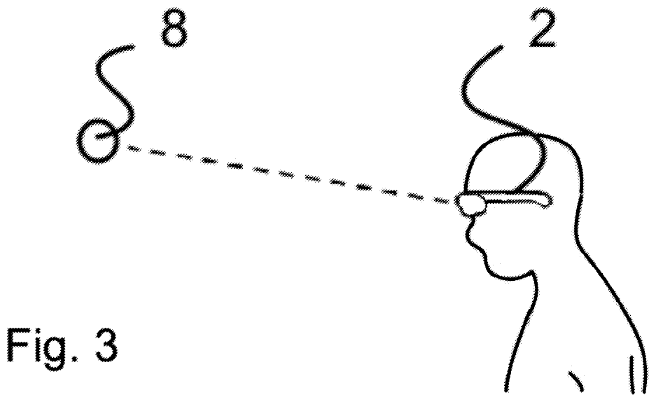

FIG. 3 is a side view of the heads-up-display module mounted to the head of a user,

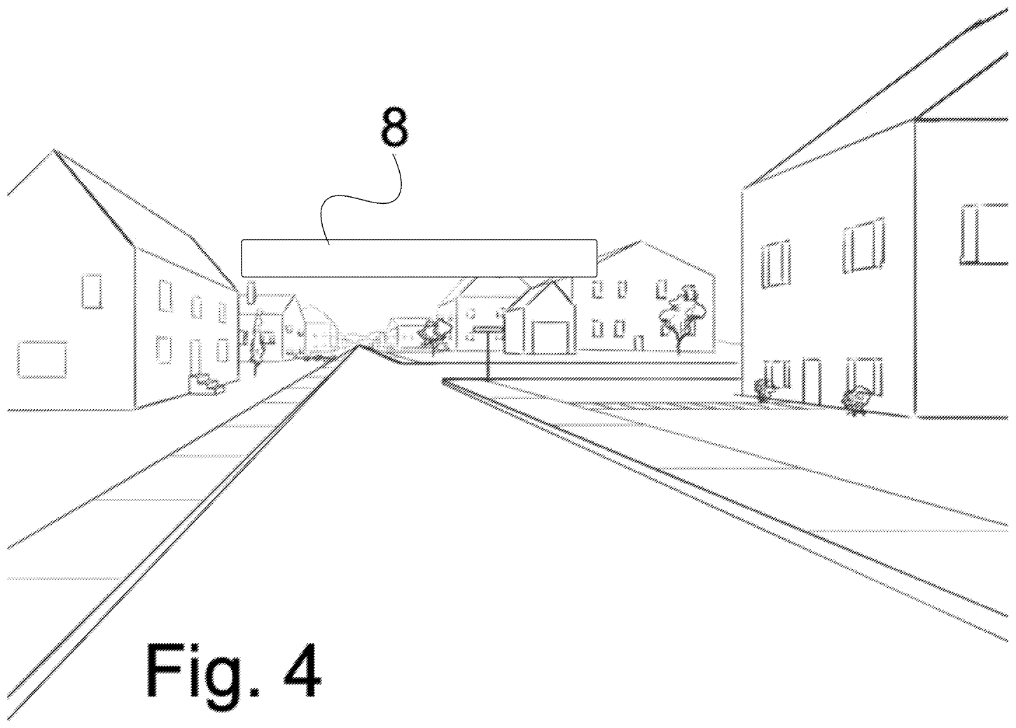

FIG. 4 shows a diagrammatic representation of a digital image experience the wearer sees when using the heads-up-display module, and

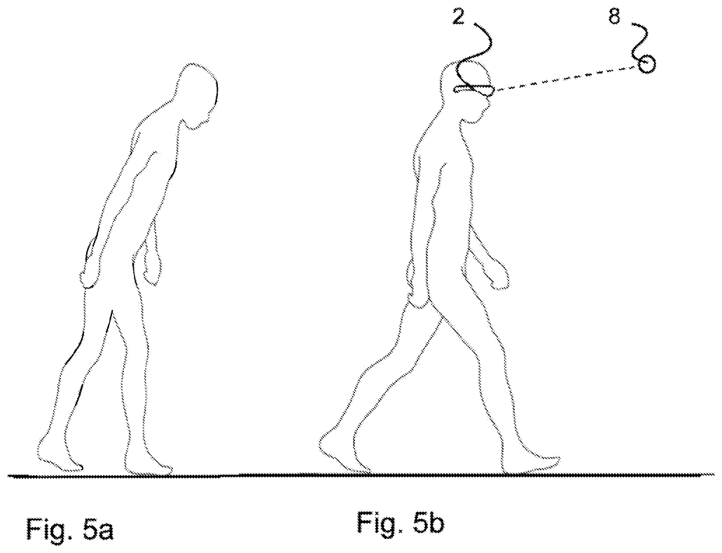

FIGS. 5a and 5b show diagrammatically, respectively, typical walking patterns and gaits of a person with Parkinson's Disease without the heads-up display module and of the same person walking in an upright manner assisted by the heads-up-display module.

Referring to FIGS. 1 and 2, a pair of eyeglasses 2 comprises an optical display system including a projecting device 4 and a display means 6. The eyeglasses 2 also comprise mounting means 8 serving to secure the eyeglasses 2 to the user. The projecting device is electrically powered by any suitable means, but preferably by one or more electrochemical cells in the form of one or more batteries housed in a compartment 7 of the eyeglasses 2 on an opposite side of a nasal bridge of the eyeglasses to the projecting device 4.

The eyeglasses 2, when worn, provide a positive impact upon the walking patterns and/or gait of people with neurodegenerative diseases such as Parkinson's Disease. The eyeglasses 2 provide a compact HUD module. The projecting device 4 preferably includes an OLED device and more preferably an AMOLED device for projecting an electronically generated image onto the display means 6.

Preferably, the optical display system is a transparent display system, the active matrix of the projecting device 4 being embedded in the field of view of the user. The display means 6 is advantageously in the form of at least one of the lenses of the eyeglasses 2, which are preferably semi-reflexive lenses that provide optimal contrast in relation to the projected image.

The projected image forms a visual cue system appearing as a digital hologram-type image which gives the illusion that it floats around 2.5 metres to around 4 metres in front of the wearer in their upper peripheral field of vision and substantially central, which greatly assists people with Parkinson's Disease, in particular, with their locomotion improving body alignment and increased stride length, therefore dramatically improving their walking and their quality of life. The image is a simple image such as a horizontal line. The image is created by software embedded within the projecting device 4.

The projected image may also take any other suitable form, such as in the form of text to provide visual messages, such as a reminder to take medication.

Referring to FIG. 3, the HUD eyeglasses 2 are being worn by a user and schematically demonstrates where in space the digital image 8 is perceived to be located by the user. Referring to FIGS. 3 and 4, the digital image 8 is focused to infinity and thus appears to the user to float, in constant focus, in space in front of them and is located in the wearer's upper peripheral field of view and substantially centrally thereof. The projection of the image can be conveniently turned on or off by the user.

Referring to FIGS. 5a and 5b, FIG. 5a shows a typical stance or gait of someone suffering from Parkinson's Disease with the characteristic stooped-over shuffling walking pattern. In contrast, in FIG. 5b, the same user wearing the eyeglasses 2 is seen to be in an upright walking position with an improved body alignment and increased stride length, therefore dramatically enhancing their walking pattern, gait, and their quality of life. In this way, posture, balance, and mobility are improved.

Advantages of using the present HUD module over Augmented Reality (AR) solutions are that:—
The HUD module requires much lower computer processing power than an AR solution, allowing the use of much smaller components.
The HUD module has a significantly longer battery life than an AR solution, allowing, importantly, all-day use for the user.
HUD modules can be produced to look identical to ordinary/normal looking eyeglasses, allowing the wearer to walk around with their walking aid being unnoticed.

It is known to provide a visual cue in a user's lower peripheral field of vision to provide an asset on the floor surface, but it has been found that a digital experience located in the wearer's upper peripheral field of vision is by far the better experience when it comes to enhancing factors such as body alignment and increased stride length.

The visual cue is displayed as a hologram-type image to be perceived to be around 2.5 metres to around 4 metres in front of the wearer and constantly in focus. By projecting a holographic-type image in this way into the upper part of the user's field of view has the effect of instantly improving posture, balance, and mobility. It is considered that by being able to identify the projected image in their upper field of view and to focus their attention upon it greatly assists users' mobility relatively instantly.

In addition to the visual cue system provided by the eyeglasses 2, an auditory stimulating means (not shown) can also be attached to the eyeglasses 2 and serving to emit an auditory signal to the user. Such auditory stimulating means may take the form of in-ear, over-ear or bone conduction headphones. Bone-conduction headphones rely on sound being transmitted through vibrations on the bones of the head and jaw and vibrations have been found to help sufferers of Parkinson's Disease. Such auditory stimulation can be in the form of repeating sound patterns and/or a voice delivering messages to the user, such as a reminder to take medication.

The user can selectively activate the projecting device 4 and/or the auditory stimulating means.

Wearing the eyeglasses 2 reduces incidents of falling and greatly improves independence, quality of life, physical activity, social interaction, mental health, mood, and even cognitive function. Wearing the eyeglasses 2 may also contribute to a reduction in reliance upon medication and also the amount of medical attention users require. For example, if the number of falls is reduced then users would require less time spent by an ambulance service and/or hospital staff attending to them. Many people with Parkinson's Disease are extremely self-conscious of their condition and how they appear different from other people, which ends up in them becoming insular, staying at home, and isolating themselves away from their community, which not only speeds up the progression of the disease but also removes their independence and puts responsibilities and pressure on their family members.

The eyeglasses 2 are practically indistinguishable from a pair of ordinary eyeglasses, allowing the wearer to walk around without their disability being easily detected as they do not attract attention as do Augmented Reality headsets. The relative anonymity suits users who do not like to attract attention to themselves.

The eyeglasses 2 provide a versatile and effective means of delivering an improvement in visual cues, in and out of the home setting, giving people the opportunity to walk and exercise outside, improving their mental health, and to live a more independent and meaningful life.

### Test Study Examples

One subject noted that when they woke up, they always walked with a shuffle, a typical Parkinson's Disease symptom, but after a few minutes of wearing the eyeglasses, the cramping they would normally experience in their toes was relieved and they were conscious of their better walking ability, which took less effort. In addition, they experienced far less aches, pains, and fatigue and was even able to walk around a shop, something which they avoided without the eyeglasses. They were also able to jog and carry things confidently.

Another subject was able to walk without a walking stick or frame with the eyeglasses as their balance was vastly improved.

In a third subject, they said every day was unpredictable, with difficulty walking and the associated falling being the main difficulties because of the problem in maintaining balance. When they used a walker frame, they felt they were bent over double which then put strain on their shoulder, which added to their discomfort. Wearing the eyeglasses gave them confidence and they walked with a straight back, larger steps and with a faster pace, drastically improving their posture. They said they had not had a fall since wearing the eyeglasses which was a major advantage for them, and their partner worried less about them when they were wearing the eyeglasses. Their episodes of freezing gait reduced by 80%.

1. Locomotion-assisting apparatus comprising: a display means, a mounting means for mounting said display means on a user; and a projection device serving to project an image onto the display means in an upper region of the display means, the image perceived by the user to appear in an upper field of view of the user and to be a distance in front of the user.

2. Locomotion-assisting apparatus according to claim 1, wherein the display means is a transparent display means.

3. Locomotion-assisting apparatus according to claim 1, wherein the projection device and display means provide an optical display system.

4. Locomotion-assisting apparatus according to claim 3, wherein the optical display system in the form of an eyeglass or pair of eyeglasses that form a heads-up display (HUD) module.

5. Locomotion-assisting apparatus according to claim 1, wherein the image is a is a horizontal line.

6. (canceled)

7. Locomotion-assisting apparatus according to claim 1, wherein the image appears in the upper field of view of the user and focused to infinity.

8. (canceled)

9. (canceled)

10. Locomotion-assisting apparatus according to claim 4, wherein the display means is at least one of the lenses of the eyeglass(es).

11. Locomotion-assisting apparatus according to claim 10, wherein the at least one lens is semi-reflexive providing optimal contrast in relation to the projected image.

12. Locomotion-assisting apparatus according to claim 1, wherein the image forms a visual cue system appearing as a digital hologram-type image which gives the illusion that it floats around 2.5 metres to around 4 metres in front of the user.

13. Locomotion-assisting apparatus according to claim 1, and further comprising an auditory stimulating means attached to the apparatus.

14. Locomotion-assisting apparatus according to claim 13, wherein the auditory stimulating means are bone-conduction headphones.

15. (canceled)

16. A method of assisting an individual with impaired locomotion, so as to improve the individual's locomotion, comprising mounting to the individual apparatus comprising a projection device and a display means and projecting an image from the projecting device on to the display means, the image being perceived by the individual to be in an upper field of view and perceived to be a distance in front of the individual.

17. (canceled)

18. (canceled)

19. A method according to claim 16, wherein the projecting includes projecting the image to appear in the upper field of view of the individual and focused to infinity.

20. (canceled)

21. (canceled)

22. A method according to claim 16, wherein the display means onto which the image is projected is at least one lens of an eyeglass or pair of eyeglasses that form a heads-up-display (HUD) module, and wherein the at least one lens is semi-reflexive providing optimal contrast in relation to the projected image.

23. A method according to claim 16, wherein the projected image forms a visual cue system appearing as a digital hologram-type image which gives the illusion that it floats around 2.5 metres to around 4 metres in front of the individual.

24. A method according to claim 16, and further comprising providing an auditory stimulation to the user.

25. (canceled)

26. A method according to claim 16, for assisting people with neurodegenerative disease with their locomotion, improving body alignment and increased stride length.

27. A method of assisting people with neurodegenerative disease by utilizing a heads-up display module on a user and projecting a hologram-type image to appear in an upper field of view of the user and to be a distance in front of the user.
